(19) United States
(12) Patent Application Publication
(10) Pub. No.: US 2025/0195819 A1
(43) Pub. Date: Jun. 19, 2025

(54) METHOD AND SYSTEM FOR CONTROLLING TURBINE MOTOR OF VENTILATOR

(71) Applicant: RESVENT MEDICAL TECHNOLOGY CO., LTD, Shenzhen (CN)

(72) Inventors: Kuan LIU, Shenzhen (CN); Rongrong ZHU, Shenzhen (CN)

(21) Appl. No.: 18/897,313

(22) Filed: Sep. 26, 2024

(30) Foreign Application Priority Data
Dec. 19, 2023 (CN) 202311762017.6

(51) Int. Cl. A61M 16/10; A61M 16/00

(52) U.S. Cl. CPC A61M 16/10; A61M 16/022

(57) ABSTRACT

A method and a system for controlling a turbine motor of a ventilator is provided. The method includes: obtaining current information and voltage information of the turbine motor; inputting the current information and the voltage information into a magnetic chain observer to obtain an angle of a turbine rotor; and performing field-oriented control on the turbine motor based on the angle of the turbine rotor. In this way, precise driving and control of the turbine motor is achieved, performance and stability of the turbine motor is improved. The magnetic chain observer has good convergence when the turbine motor is at a lower rotational speed, and the turbine motor can be started directly in a closed-loop manner without positioning or being in an open-loop manner. Therefore, time consumed for starting the turbine motor is reduced, and reliability of the starting is improved.

### CROSS-REFERENCE TO RELATED APPLICATION

This application claims priority to Chinese Patent Application No. 202311762017.6, titled “METHOD AND SYSTEM FOR CONTROLLING TURBINE MOTOR OF VENTILATOR” and filed to the China National Intellectual Property Administration on Dec. 19, 2023, the entire contents of which are incorporated herein by reference.

### TECHNICAL FIELD

The present disclosure relates to the field of ventilators, and in particular to a method and a system for controlling a turbine motor of a ventilator.

### BACKGROUND

In modern clinical medicine, a ventilator, as an effective means that replaces autonomous ventilation, has been commonly used in treating patients who suffer from obstructive sleep apnea and chronic obstructive pulmonary disease (COPD). The ventilator occupies a very important position in the field of modern medicine.

When the ventilator is operating normally, a turbine motor is configured to provide air to the ventilator. A Hall sensor is configured in the turbine motor in the ventilator. When the turbine motor is driven, six-step commutation control or field-oriented control is usually performed through an angle detected by the Hall sensor. However, in the above method, three Hall sensors are needed, resulting in torque pulsation in the turbine motor during commutation, such that undesired vibration and noise are introduced. In addition, the above turbine driving method relies significantly on Hall signals. In practice, due to external interference, noise may be present on the Hall signals, resulting in failure in turbine motor wheel commutation, causing the turbine motor to stall or to be damaged.

### SUMMARY OF THE DISCLOSURE

To solve the above technical problem, the present disclosure provides a method and a system for controlling a turbine motor of a ventilator.

In a first aspect, the present disclosure provides a method for controlling a turbine motor of a ventilator, including:
obtaining current information and voltage information of the turbine motor;
inputting the current information and the voltage information into a magnetic chain observer to obtain an angle of a turbine rotor; and
performing field-oriented control on the turbine motor based on the angle of the turbine rotor.

In some embodiments, operation of inputting the current information and the voltage information into the magnetic chain observer to obtain the angle of the turbine rotor, includes:
establishing, based on a basic configuration and an operating principle of the turbine motor, a mathematical model of the turbine motor in a stationary coordinate system;
establishing, based on the mathematical model, a state spatial equation of the magnetic chain observer; and
obtaining the angle of the turbine rotor by performing calculation based on the state spatial equation.

In some embodiments, the mathematical model of the turbine motor in the stationary coordinate system is as follows:

$$\dot{L {ι}_{αβ}} = - {R}_{s} {i}_{αβ} + ω {ψ}_{m} [ \begin{matrix}sin θ \\ - cos θ\end{matrix} ] + {v}_{αβ}$$

$${T}_{e} = \frac{3 P}{4} {ψ}_{m} ( {i}_{α} cos θ - {i}_{β} sin θ )$$

T The i_{αβ} denotes a stator current, i_{αβ}=[i_{α}′, i_{β}]; the v_{αβ} denotes a phase voltage, v_{αβ}=[v_{α}>v_{β}]^{T}; the θ denotes an angle; the ω denotes an electrical rotational speed; the Rₛ denotes a stator resistance; the L denotes a stator inductance; the ψₘ denotes a magnetic chain of a permanent magnet; the Tₑ denotes an electromagnetic torque; and the P denotes the number of motor poles.

In some embodiments, the operation of establishing, based on the mathematical model, the state spatial equation of the magnetic chain observer, includes:
defining a new state variable, simplifying the mathematical model, and obtaining a simplified state spatial model;
defining a vector function, and establishing the state spatial equation for the magnetic chain observer based on the vector function.

In some embodiments, the simplified state spatial model is as follows:

$$\dot{x} = \dot{L {ι}_{αβ}} - {ωψ}_{m} [ \begin{matrix}sin θ \\ - cos θ\end{matrix} ] = y$$

The \dot{x} denotes a first order derivative of x, the x denotes a new state vector,

$$x = L {i}_{αβ} + {ψ}_{m} [ \begin{matrix}cos θ \\ sin θ\end{matrix} ] , y ≡ - {R}_{s} {i}_{αβ} + {v}_{αβ} ;$$

the i_{αβ} denotes the stator current, i_{αβ}=[i_{α}, i_{β}], the θ denotes the angle, the ω denotes the electrical rotational speed, the L denotes the stator inductance, the L denotes the stator inductance, and the Um denotes the magnetic chain of the permanent magnet.

The state spatial equation of the magnetic chain observer is as follows:

$$\dot{\hat{x}} = y + \frac{γ}{2} η ( \hat{x} ) [ {ψ}_{m}^{2} - {‖ η ( \hat{x} ) ‖}^{2} ]$$

The \dot{x} denotes the state variable of the magnetic chain observer; the γ denotes a gain of the magnetic chain observer; the ψₘ²−∥η(\hat{x})∥² denotes a square of a distance between η(\hat{x}) and a circle having a radius of ψₘ; and the η(\hat{x}) denotes the vector function.

In some embodiments, the operation of obtaining the angle of the turbine rotor by performing calculation based on the state spatial equation, includes:
obtaining an estimated value of the angle of the turbine rotor by performing calculation by an inverse tangent function; or obtaining an estimated value of the angle of the turbine rotor by performing calculation by a phase lock loop.

In some embodiments, the operation of obtaining the estimated value of the angle of the turbine rotor by performing calculation by the inverse tangent function, includes:
obtaining the estimated value of the angle of the turbine rotor by performing calculation by the inverse tangent function; and
calculating a target rotational speed based on the estimated value of the angle of the turbine rotor.

The operation of performing field-oriented control on the turbine motor based on the angle of the turbine rotor, includes:
performing field-oriented control on the turbine motor based on the angle of the turbine rotor and the target rotational speed.

In some embodiments, the estimated value \hat{θ} of the angle of the turbine rotor is calculated based on the following equation:

$$\hat{θ} = {tan}^{- 1} ( \frac{- L {i}_{β}}{- L {i}_{α}} )$$

The x is the state variable, and

$$x = [ \begin{matrix}{x}_{1} \\ {x}_{2}\end{matrix} ] ;$$

each of the i_{α} and the i_{β} denotes the stator current, the L denotes the stator inductance.

In some embodiments, the operation of obtaining the estimated value of the angle of the turbine rotor by performing calculation by the phase lock loop, includes:
obtaining the estimated value of the angle of the turbine rotor and the target rotational speed by performing calculation by the phase lock loop.

The operation of performing field-oriented control on the turbine motor based on the angle of the turbine rotor, includes:
performing field-oriented control on the turbine motor based on the angle of the turbine rotor and the target rotational speed.

In a second aspect, the present disclosure provides a method for controlling a turbine motor of a ventilator, including: the method of controlling the turbine motor of the ventilator according to any of the above embodiments.

According to the present disclosure, the method and the system for controlling the turbine motor of the ventilator are provided. The angle of the turbine rotor is calculated by the magnetic chain observer, and field-oriented control is performed, according to the angle of the turbine rotor, on the turbine motor. In this way, precise driving and control of the turbine motor is achieved, performance and stability of the turbine motor is improved. The magnetic chain observer has good convergence when the turbine motor is at a lower rotational speed, and the turbine motor can be started directly in a closed-loop manner without positioning or being in an open-loop manner. Therefore, time consumed for starting the turbine motor is reduced, and reliability of the starting is improved.

### BRIEF DESCRIPTION OF THE DRAWINGS

In order to more clearly illustrate the technical solutions of the embodiments of the present disclosure, the accompanying drawings for describing the embodiments will be briefly introduced in the following. It is understood that the following accompanying drawings only show some embodiments of the present application, and therefore, shall not be regarded as a limitation of the scope of the present disclosure. Any ordinary skilled person in the art may obtain other accompanying drawings based on these drawings without making any creative work.

FIG. 1 is a flow chart of a method for controlling a turbine motor of a ventilator according to some embodiments of the present disclosure.

FIG. 2 is a flow chart of an operation S200 of the method according to some embodiments of the present disclosure.

FIG. 3 is a flow chart of an operation S220 of the method according to some embodiments of the present disclosure.

FIG. 4 is a flow chart of an operation S240 of the method according to some embodiments of the present disclosure.

FIG. 5 is a structural schematic diagram of a phase lock loop according to some embodiments of the present disclosure.

### DETAILED DESCRIPTION

In order to make the objectives, technical solutions and advantages of the embodiments of the present disclosure clearer, the technical solutions in the embodiments of the present disclosure will be described clearly and completely in the following by referring to the accompanying drawings in the embodiments of the present disclosure. Obviously, the described embodiments are a part of, not all of, the embodiments of the present disclosure. Components of the described and illustrated embodiments shown in the accompanying drawings herein may be arranged and designed in a variety of different configurations.

Accordingly, the following detailed description of the embodiments shown in the accompanying drawings of the present disclosure is not intended to limit the claimed scope of the present disclosure, but merely indicates selected embodiments of the present disclosure. All other embodiments, which are obtained by any ordinary skilled person in the art based on the embodiments of the present disclosure without making creative work, shall fall within the scope of the present disclosure.

To be noted that similar reference numerals and letters indicate similar items in the following accompanying drawings. Therefore, once an item is defined in one accompanying drawing, the item may not be further defined and explained in subsequent accompanying drawings.

In the description of the present disclosure, to be noted that the terms “up”, “down” and the so on, are used to indicate an orientation or a positional relationship based on the orientation or the positional relationship shown in the accompanying drawings or indicate an orientation or a positional relationship in which the product of the present disclosure is commonly placed when being in use. The terms are used only for the purpose of facilitating the description of the present disclosure and simplifying the description and shall not indicate or imply that the devices or elements referred to must have a particular orientation or must be configured and operated in a particular orientation. Therefore, the terms shall not be interpreted as a limitation of the present disclosure. Furthermore, the terms “first”, “second”, and so on, are used only to distinguish descriptions and shall not be interpreted as indicating or implying relative importance.

In addition, the terms “horizontal”, “vertical”, “overhanging”, and so on, do not mean that the components are required to be absolutely horizontal or overhanging but may be slightly inclined. For example, “horizontal” simply means that an orientation is more horizontal than “vertical”, and does not mean that a structure must be perfectly horizontal, and the structure may be slightly inclined.

In the description of the present disclosure, to be noted that the terms “configured”, “mounted”, “inter-connected”, and “connected” shall be interpreted in a broad sense, unless otherwise expressly specified and qualified. For example, “connection” may be fixed connection, detachable connection, or being connected to form a one-piece structure; mechanical connection, or electrical connection; direct connection, or indirect connection through an intermediate medium; or internal connection of two elements. Any ordinary skilled person in the art shall understand the specific meaning of the above terms in the present disclosure case by case.

To be noted that different features in the embodiments of the present disclosure may be combined with each other without conflict.

The present disclosure provides a technical solution to solve the above technical problem, specific embodiments of the present disclosure are described in detail below by referring to the accompanying drawings.

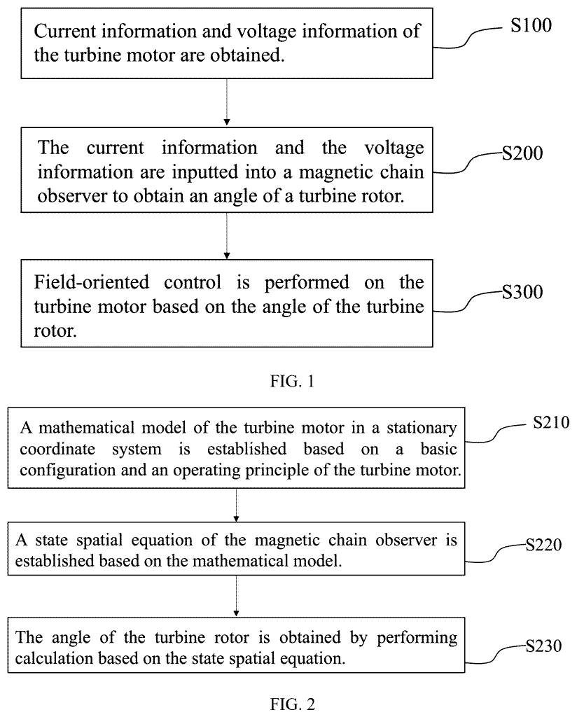

As shown in FIG. 1, the present disclosure provides a method for controlling a turbine motor of a ventilator. The method is applied to a ventilator. The method for controlling the turbine motor of the ventilator includes the following operations.

In an operation S100, current information and voltage information of the turbine motor are obtained.

In the present embodiment, the current information of the turbine motor is obtained by a sensor performing sampling, and the voltage information is obtained by a microcontroller (MCU) performing calculation through field-oriented control. Specifically, the current information includes a stator current of an α-axis and a stator current of a β-axis. The voltage information includes a phase voltage of the α-axis and a phase voltage of the β-axis.

The turbine motor may be a surface-mounted permanent magnet synchronous motor (SPMSM). The turbine motor does not need any Hall sensor. Compared to a motors configured with the Hall sensor, the turbine motor in the present disclosure operates without depending on Hall signals, a turbine torque is smoother, and noise and vibration are reduced.

In an operation S200, the current information and the voltage information are inputted into a magnetic chain observer to obtain an angle of a turbine rotor.

In the present embodiment, the current information and the voltage information obtained in the operation S100 are inputted into the magnetic chain observer to obtain a continuous angle of the turbine rotor.

Specifically, the magnetic chain observer may be a nonlinear magnetic chain observer. By applying suitable configuration to the nonlinear magnetic chain observer and adjusting parameters of the nonlinear magnetic chain observer, the angle of the turbine rotor can be estimated accurately. In this way, a high-performance control of the turbine motor can be achieved.

The inventor discovers, based on research, that in a conventional induction motor or a conventional permanent magnet synchronous motor, no sensor is configured to directly obtain position information of the rotor, and therefore, a closed-loop control cannot be achieved when the turbine motor is operating at low speeds or in a standstill state. However, in the present disclosure, the magnetic chain observer is configured to estimate, in real time, the angle of the turbine rotor based on the stator current and magnetic chain information of the motor. In this way, the closed-loop control can be achieved when the turbine motor is operating at low speeds or even at zero speed.

In an operation S300, field-oriented control is performed on the turbine motor based on the angle of the turbine rotor.

In the present embodiment, the field-oriented control (FOC) is performed on the turbine motor based on the angle of the turbine rotor obtained at the operation S200. That is, precise control of the turbine motor is achieved by controlling the magnetic field and the current of the turbine motor, enabling the turbine torque to be smoother and enabling the noise and the vibration to be reduced.

Specifically, according to space vector pulse width modulation (SVPWM), a three-phase pulse width modulation (PWM) duty cycle is calculated based on the angle of the turbine rotor. In this way, the turbine motor is controlled precisely to improve an efficiency and a response speed of the turbine motor.

In the above embodiment, the magnetic chain observer is configured to calculate the angle of the turbine rotor, such that the field-oriented control is performed on the turbine motor based on the angle of the turbine rotor. In this way, precise driving and control of the turbine motor is achieved, the performance and the stability of the turbine motor are improved. The magnetic chain observer has good convergence when the turbine motor is at a lower rotational speed, and the turbine motor can be started directly in a closed-loop manner without positioning or being in an open-loop manner. Therefore, time consumed for starting the turbine motor is reduced, and reliability of the starting is improved

In an implementation, as shown in FIG. 2, the operation S200 includes the following sub-operations.

In a sub-operation S210, a mathematical model of the turbine motor in a stationary coordinate system is established based on a basic configuration and an operating principle of the turbine motor.

In the present embodiment, the turbine motor may be the SPMSM. According to a basic configuration and an operating principle of the SPMSM, a mathematical model of the turbine motor in a two-phase stationary coordinate system α-β is established.

The mathematical model of the turbine motor in the stationary coordinate system is as follows:

$$\begin{matrix}\dot{L {ι}_{a β}} = - {R}_{s} {i}_{αβ} + ω {ψ}_{m} [ \begin{matrix}sin θ \\ - cos θ\end{matrix} ] + {v}_{a β} \\ {T}_{e} = \frac{3 P}{4} {ψ}_{m} ( {i}_{α} cos θ - {i}_{β} sin θ )\end{matrix}$$

The i_{αβ} denotes the stator current, i_{αβ}=[i_{α}, i_{β}]^{T}; the [ . . . ]^{T} denotes transpose of a matrix; the i_{α} denotes a stator current of the α-axis; the i_{β} denotes a stator current of the β-axis; the v_{αβ} denotes the phase voltage, v_{αβ}=[v_{α}, v_{β}]^{T}; the v_{α} denotes a phase voltage of the α-axis; the v_{β} denotes a phase voltage of the β-axis; the θ denotes the angle; the ω denotes an electrical rotational speed; the Rₛ denotes a stator resistance; the L denotes a stator inductance; the ψₘ denotes a magnetic chain of the permanent magnet; the Tₑ denotes an electromagnetic torque; and the P denotes the number of motor poles.

In the mathematical model of the turbine motor, if the rₛ denotes a phase resistance and the lₛ denotes a phase inductance,

$${R}_{s} = \frac{3}{2} {r}_{s} , and L = \frac{3}{2} {l}_{s} .$$

In the SPMSM, a quadrature (Q)-axial inductance is equal to a direct (D)-axial inductance. Therefore, a value of the stator inductance L is independent of the angle θ. In addition, the stator current i_{αβ} can be obtained from a measurement, and the phase voltage v_{αβ} is known, and therefore, only the angle θ and the electrical rotational speed @ need to be calculated.

In an operation S220, a state spatial equation of the magnetic chain observer is established based on the mathematical model.

In the present embodiment, the state spatial equation of the magnetic chain observer is established based on the mathematical model obtained at the operation S210, and the magnetic chain observer is configured to describe a state change of the motor.

In an operation S230, the angle of the turbine rotor is obtained by performing calculation based on the state spatial equation.

In the present embodiment, the current information and the voltage information of the turbine motor obtained at the operation S100 are inputted into the state spatial equation to obtain an estimated value of the angle of the turbine rotor. In this way, precise control of the turbine motor is achieved.

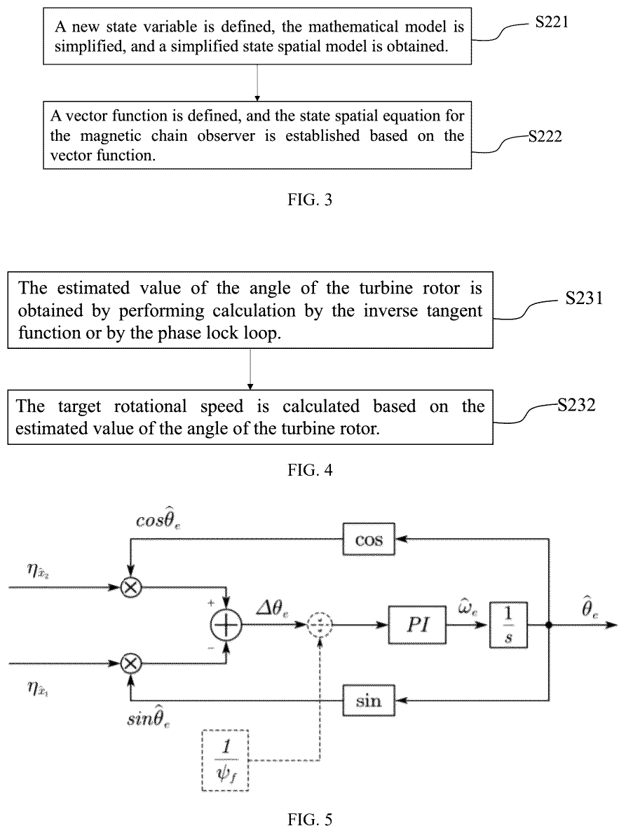

In an implementation, as shown in FIG. 3, the operation S220 includes the following sub-operations.

In a sub-operation S221, a new state variable is defined, the mathematical model is simplified, and a simplified state spatial model is obtained.

In the present embodiment, the new state variable

$$x = [ \begin{matrix}{x}_{1} \\ {x}_{2}\end{matrix} ]$$

is defined, where

$$x = L {i}_{αβ} + {ψ}_{m} [ \begin{matrix}cos θ \\ sin θ\end{matrix} ] ,$$

and y≡−Rₛi_{αβ}+v_{αβ}. Therefore, the simplified state spatial model is as follows:

$$\dot{x} = \dot{L {ι}_{αβ}} - {ωψ}_{m} [ \begin{matrix}sin θ \\ - cos θ\end{matrix} ] = y$$

The \dot{x} denotes a first order derivative of x, the x denotes a new state vector, the y denotes an inverse electrical potential, the i_{αβ} denotes the stator current, i_{αβ}=[i_{α}, i_{β}]^{T}, the θ denotes the angle, the ω denotes the electrical rotational speed, the L denotes the stator inductance, and the ψₘ denotes the magnetic chain of the permanent magnet.

In a sub-operation S222, a vector function is defined, and the state spatial equation for the magnetic chain observer is established based on the vector function.

In the present embodiment, the vector function η(\hat{x})=x−Li_{αβ} is defined. A modulus of the vector function is equal to a magnitude of the magnetic chain. That is, ∥η(\hat{x})∥²=ψₘ². The established state spatial equation of the nonlinear magnetic chain observer is as follows:

$$\dot{\hat{x}} = y + \frac{γ}{2} η ( \hat{x} ) [ {ψ}_{m}^{2} - {‖ η ( \hat{x} ) ‖}^{2} ]$$

The \dot{\hat{x}} denotes a state variable of the observer, where \hat{x}ϵR², and the R is a real number. The γ denotes a gain of the observer. The ψₘ²−∥η(\hat{x})∥² denotes a square of a distance between η(\hat{x}) and a circle having a radius of ψₘ. The η(\hat{x}) denotes the vector function.

In an implementation, when the angle of the turbine rotor according to the state spatial equation, the estimated value of the angle of the turbine rotor and a target rotational speed are obtained by means of an inverse tangent function or a phase lock loop (PLL). In this way, the field-oriented control of the turbine motor is achieved.

In an implementation, as shown in FIG. 4, the operation S230 includes the following sub-operations.

In a sub-operation S231, the estimated value of the angle of the turbine rotor is obtained by performing calculation by the inverse tangent function or by the phase lock loop.

In the present embodiment, when the inverse tangent function is to be used for calculation, the inverse tangent function is obtained based on the state spatial equation, and therefore, the estimated value of the angle of the turbine rotor can be calculated based on the inverse tangent function.

Specifically, the estimated value \hat{θ} of the angle of the turbine rotor is calculated based on the following equation:

$$\hat{θ} = {tan}^{- 1} ( \frac{- {Li}_{β}}{- {Li}_{α}} )$$

The x denotes the state variable,

$$x = [ \begin{matrix}{x}_{1} \\ {x}_{2}\end{matrix} ] ;$$

the i_{α} denotes the stator current in the α-axis; the i_{β} denotes the stator current in the β-axis; and the L denotes the stator inductance.

In a sub-operation S232, the target rotational speed is calculated based on the estimated value of the angle of the turbine rotor.

In the present embodiment, the target rotational speed is calculated based on the estimated value \hat{θ} of the angle of the turbine rotor obtained at the operation S231.

When performing the field-oriented control of the turbine motor based on the angle of the turbine rotor, the field-oriented control of the turbine motor is performed based on the estimated value \hat{θ} of the angle of the turbine rotor and the target rotational speed.

In an implementation, as shown in FIG. 5, when calculating the estimated value of the angle of the turbine rotor based on the phase lock loop, the estimated value of the angle of the turbine rotor and target rotational speed can be obtained through the phase lock loop.

When performing the field-oriented control on the turbine motor according to the angle of the turbine rotor, the field-oriented control is performed, based on the estimated value of the angle of the turbine rotor and target rotational speed, on the turbine motor.

The present disclosure further provides a system for controlling the turbine motor of the ventilator. The system includes the method for controlling the turbine motor of the ventilator as described in any of the above embodiments.

In summary, the present disclosure provides the method and the system for controlling the turbine motor of the ventilator. The angle of the turbine rotor is calculated by the magnetic chain observer, and field-oriented control is performed, according to the angle of the turbine rotor, on the turbine motor. In this way, precise driving and control of the turbine motor is achieved, performance and stability of the turbine motor is improved. The magnetic chain observer has good convergence when the turbine motor is at a lower rotational speed, and the turbine motor can be started directly in a closed-loop manner without positioning or being in an open-loop manner. Therefore, time consumed for starting the turbine motor is reduced, and reliability of the starting is improved.

To be noted that, in the present disclosure, relational terms, such as first and second, are used only to distinguish one entity or one operation from another, and do not necessarily require or imply existence of any actual relationship or order between the entities or operations. Furthermore, the terms “include”, “contain”, and any other variant thereof, are intended to cover non-exclusive inclusion. Therefore, a process, a method, an article, or an apparatus including a set of elements includes not only the listed elements, but further includes other elements that are not expressly listed or other elements that are inherently included in the process, the method, the article or the apparatus. Without further limitation, elements defined by the phrase “include . . . ” do not preclude existence of additional identical elements in the process, the method, the article or the apparatus.

The above description describes only preferred embodiments of the present disclosure and does not limit the present disclosure. For any ordinary skilled person in the art, the present disclosure may have changes and variations. Any modifications, equivalent substitutions, and improvements made within the concept and principles of the present disclosure shall be included in the scope of the present disclosure.

What is claimed is:

1. A method for controlling a turbine motor of a ventilator, comprising:
obtaining current information and voltage information of the turbine motor;
inputting the current information and the voltage information into a magnetic chain observer to obtain an angle of a turbine rotor; and
performing field-oriented control on the turbine motor based on the angle of the turbine rotor.

2. The method according to claim 1, wherein the operation of inputting the current information and the voltage information into the magnetic chain observer to obtain the angle of the turbine rotor, comprises:
establishing, based on a basic configuration and an operating principle of the turbine motor, a mathematical model of the turbine motor in a stationary coordinate system;
establishing, based on the mathematical model, a state spatial equation of the magnetic chain observer; and
obtaining the angle of the turbine rotor by performing calculation based on the state spatial equation.

3. The method according to claim 2, wherein, the mathematical model of the turbine motor in the stationary coordinate system is as follows:
$$\begin{matrix}\dot{L {ι}_{αβ}} = - {R}_{s} {i}_{αβ} + ω {ψ}_{m} [ \begin{matrix}sin θ \\ - cos θ\end{matrix} ] + {v}_{αβ} \\ {T}_{e} = \frac{3 P}{4} {ψ}_{m} ( {i}_{α} cos θ - {i}_{β} sin θ )\end{matrix}$$
wherein the i_{αβ} denotes a stator current, i_{αβ}=[i_{α}, i_{β}]^{T}; the v_{αβ} denotes a phase voltage, v_{αβ}=[v_{α}, v_{β}]^{T}; the θ denotes an angle; the ω denotes an electrical rotational speed; the Rₛ denotes a stator resistance; the L denotes a stator inductance; the ψₘ denotes a magnetic chain of a permanent magnet; the Tₑ denotes an electromagnetic torque; and the P denotes the number of motor poles.

4. The method according to claim 2, wherein, the operation of establishing, based on the mathematical model, the state spatial equation of the magnetic chain observer, comprises:
defining a new state variable, simplifying the mathematical model, and obtaining a simplified state spatial model;
defining a vector function, and establishing the state spatial equation for the magnetic chain observer based on the vector function.

5. The method according to claim 4, wherein, the simplified state spatial model is as follows:
$$\dot{x} = \dot{L {ι}_{αβ}} - {ωψ}_{m} [ \begin{matrix}sin θ \\ - cos θ\end{matrix} ] = y$$
wherein, the \dot{x} denotes a first order derivative of x, the x denotes a new state vector
$$x = \dot{L {ι}_{αβ}} + {ψ}_{m} [ \begin{matrix}cos θ \\ sin θ\end{matrix} ] , y ≡ - {R}_{s} {i}_{αβ} + {v}_{αβ} ;$$
the i_{αβ} denotes the stator current, i_{α⊕}=[i_{α}, i_{β}]^{T}, the θ denotes the angle, the ω denotes the electrical rotational speed, the L denotes the stator inductance, the L denotes the stator inductance, and the ψₘ denotes the magnetic chain of the permanent magnet;
the state spatial equation of the magnetic chain observer is as follows:
$$\dot{\hat{x}} = y + \frac{γ}{2} η ( \hat{x} ) [ {ψ}_{m}^{2} - {‖ η ( \hat{x} ) ‖}^{2} ]$$
wherein, the \dot{\hat{x}} denotes the state variable of the magnetic chain observer; the γ denotes a gain of the magnetic chain observer; the ψₘ²−∥η(\hat{x})∥² denotes a square of a distance between η(\hat{x}) and a circle having a radius of ψₘ; and the η(\hat{x}) denotes the vector function.

6. The method according to claim 2, wherein, the operation of obtaining the angle of the turbine rotor by performing calculation based on the state spatial equation, comprises:
obtaining an estimated value of the angle of the turbine rotor by performing calculation by an inverse tangent function;
or
obtaining an estimated value of the angle of the turbine rotor by performing calculation by a phase lock loop.

7. The method according to claim 6, wherein,
the operation of obtaining the estimated value of the angle of the turbine rotor by performing calculation by the inverse tangent function, comprises:
obtaining the estimated value of the angle of the turbine rotor by performing calculation by the inverse tangent function; and
calculating a target rotational speed based on the estimated value of the angle of the turbine rotor;
the operation of performing field-oriented control on the turbine motor based on the angle of the turbine rotor, comprises:
performing field-oriented control on the turbine motor based on the angle of the turbine rotor and the target rotational speed.

8. The method according to claim 7, wherein, the estimated value \hat{θ} of the angle of the turbine rotor is calculated based on the following equation:
$$\hat{θ} = {tan}^{- 1} ( \frac{- {Li}_{β}}{- {Li}_{α}} )$$
wherein the x is the state variable, and
$$x = [ \begin{matrix}{x}_{1} \\ {x}_{2}\end{matrix} ] ;$$
each of the i_{α} and the i_{β} denotes the stator current, the L denotes the stator inductance.

9. The method according to claim 6, wherein,
the operation of obtaining the estimated value of the angle of the turbine rotor by performing calculation by the phase lock loop, comprises:
obtaining the estimated value of the angle of the turbine rotor and the target rotational speed by performing calculation by the phase lock loop;
the operation of performing field-oriented control on the turbine motor based on the angle of the turbine rotor, comprises:
performing field-oriented control on the turbine motor based on the angle of the turbine rotor and the target rotational speed.

10. A system for controlling a turbine motor of a ventilator, comprising the method of controlling the turbine motor of the ventilator according to claim 1.
